(19) United States
(12) Patent Application Publication
(10) Pub. No.: US 2009/0259157 A1
(43) Pub. Date: Oct. 15, 2009

(54) Method for imparting antimicrobial characteristics to hydrophilic fabrics

(76) Inventor: Tom Thomas, Summerville, SC (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 12/082,757

(22) Filed: Apr. 14, 2008

(51) Int. Cl. A61L 15/44; B05D 3/00

(52) U.S. Cl. 602/48; 427/2.31

(57) ABSTRACT

The present invention is directed to a method for imparting antimicrobial characteristics to hydrophilic fabrics. The method involves: (1) providing a hydrophilic fabric; (2) providing a first solution comprising at least one oligodynamic metal ion, wherein the first solution is free of basic nitrogen compounds; (3) contacting the hydrophilic fabric with the first solution to allow the hydrophilic fabric to absorb metal ions from the first solution; and (4) precipitating or reducing the metal ions that have been absorbed by the fabric so that the hydrophilic fabric contains an oligodynamic metal salt or free oligodynamic metal and has antimicrobial characteristics. The invention is also directed to disposable medical cloths having antimicrobial characteristics.

### BACKGROUND OF THE INVENTION

### Field of the Invention

The present invention relates generally to a method for imparting antimicrobial characteristics to hydrophilic fabrics and fabrics made therefrom.

### SUMMARY OF THE INVENTION

In an embodiment, the present invention is directed to a novel method for imparting antimicrobial characteristics to hydrophilic fabrics. The method involves: (1) providing a hydrophilic fabric; (2) providing a first solution comprising at least one oligodynamic metal ion, wherein the first solution is free of basic nitrogen compounds; (3) contacting the hydrophilic fabric with the first solution to allow the hydrophilic fabric to absorb metal ions from the first solution; and (4) precipitating the metal ions that have been absorbed by the fabric so that the hydrophilic fabric contains an oligodynamic metal salt and has antimicrobial characteristics.

In another embodiment, instead of precipitating the metal ions so that the hydrophilic fabric contains an oligodynamic metal salt, the method involves reducing the metal ions that have been absorbed by the fabric so that the hydrophilic fabric contains free oligodynamic metal and has antimicrobial characteristics. In other embodiments, the invention is directed to fabrics, including disposable medical cloths, produced according to one of the methods set forth herein.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

Briefly, the invention is directed to a method for imparting antimicrobial characteristics to hydrophilic fabrics. In an embodiment, the method involves the steps of: (1) providing a hydrophilic fabric; (2) providing a first solution of at least one oligodynamic metal ion, wherein the first solution is free of basic nitrogen compounds; (3) contacting the hydrophilic fabric with the first solution to allow the hydrophilic fabric to absorb metal ions from the first solution; and (4) precipitating the metal ions that have been absorbed by the fabric so that the hydrophilic fabric contains a oligodynamic metal salt and has antimicrobial characteristics.

The term “fabric,” as used herein, means any sheet-like or web material which is formed, in whole or in part, from a plurality of fibers. In an embodiment of the present invention, the fabric may be a woven or a non-woven fabric. The term “woven fabric” means a fabric having individual fibers interlaced in a regular order. The term “nonwoven fabric” means a fabric having a structure of individual fibers which are interlaid, but not in a regular manner. Nonwoven fabrics have been formed using many processes such as, for example, melt blowing processes, spunbonding processes, and bonded carded web processes.

The fabrics of the present invention will generally be hydrophilic in nature so that they can absorb the necessary antimicrobial agents. In addition, the hydrophilicity allows the fabrics to act as towels, wipes, or absorbent garments in certain embodiments. As used herein, the terms “hydrophilic” or “hydrophilic fabric” are used to describe fabrics that are water-absorbent. More specifically, the fabrics are water-absorbent to an extent that allows a sufficient amount of metal ions to be absorbed into the fabric to create a final product that will act as an antimicrobial fabric for the particular antimicrobial purpose intended for that fabric. The fabrics of the present invention may contain only fibers that are hydrophilic or may contain some fibers that are hydrophilic and others that are hydrophobic. The degree of hydrophilicity will vary, depending on the particular use of the fabric and the level of antimicrobial activity needed.

In some embodiments, the hydrophilic fabric may be made from a natural fiber such as cotton, wool, linen, jute, hemp, silk, or cellulosic pulp fibers, such as wood pulp. In other embodiments, the hydrophilic fabric may be made from synthetic fibers that are either naturally hydrophilic or which are modified to become hydrophilic. Synthetic fibers useful in the present invention may include, but are not limited to, rayon fibers, polyvinyl alcohol fibers, ethylene vinyl alcohol copolymer fibers, and various polyolefin fibers.

Suitable polymeric fibers for use in the present invention include, but are not limited to, fibers made from polyolefins, polyesters, polyamides, and copolymers and blends thereof. Polyolefins suitable for the fibers include polyethylene, e.g., high density polyethylene, medium density polyethylene, low density polyethylene, and linear low density polyethylene; polypropylene, e.g., isotactic polypropylene, syndiotactic polypropylene, blends thereof, and blends of isotactic polypropylene and atactic polypropylene; polybutylene, e.g., poly(1-butene) and poly(2-butene); polypentene, e.g., poly(1-pentene) and poly(2-pentene); poly(3-methyl-1-pentene); poly(4-methyl-1-pentene); and copolymers and blends thereof.

Suitable copolymers include random and block copolymers prepared from two or more different unsaturated olefin monomers, such as ethylene/propylene and ethylene/butylene copolymers. Polyamides suitable for the fibers include nylon 6, nylon 6/6, nylon 4/6, nylon 11, nylon 12, nylon 6/10, nylon 6/12, nylon 12/12, copolymers of caprolactam and alkaline oxide diamine, and the like, as well as blends and copolymers thereof. Suitable polyesters include polyethylene terephthalate, polybutylene terephthalate, polytetramethylene terephthalate, polycyclohexylene-1,4-dimethylene terephthalate, and isophthalate copolymers thereof, as well as blends thereof.

In other embodiments, the hydrophilic fabric may be made from a combination or blend of natural and synthetic fibers. The hydrophilic fabric of the invention may also include any natural or synthetic fibers, such as those listed above, that have been treated or otherwise altered to cause them to become hydrophilic or partially hydrophilic.

In some embodiments, the hydrophilic fabric of the invention may be a bicomponent fiber. Bicomponent fibers are multicomponent fibers wherein two fibers having differing characteristics are combined into a single fiber. Bicomponent fibers generally have a core and sheath structure in which the core is a polyester and the sheath is a polyolefin. Other bicomponent fiber structures, however, may also be utilized. For example, bicomponent fibers may be formed with the two components residing in various side-by-side relationships as well as concentric and eccentric core and sheath configurations.

In one embodiment, the hydrophilic fabric of the invention may be a blend of cotton and polyester. In this embodiment, cotton may comprise between 10% and 50% of the hydrophilic fabric. In another embodiment, cotton may comprise between about 20% and 40% of the hydrophilic fabric. In yet another embodiment, cotton may comprise about 35% of the hydrophilic fabric. In an embodiment, polyester may comprise between about 50% and 85% of the hydrophilic fabric. In another embodiment, polyester may comprise between about 60% and 75% of the hydrophilic fabric. In yet another embodiment, polyester may comprise about 65% of the hydrophilic fabric. In a particular embodiment, the hydrophilic fabric comprises about 35% cotton and about 65% polyester.

In another embodiment, the hydrophilic fabric comprises a composite of wood pulp and spunbonded polyester. In this embodiment, the wood pulp may be derived from softwood trees such as spruce, pine, fir, larch or hemlock, or hardwood trees such as eucalyptus, aspen or birch. The wood pulp may be a mixture of different types and/or qualities of pulp fibers. For example, the invention may include a pulp containing a portion of low-average fiber length pulp and a portion of high-average fiber length pulp. The pulp may contain secondary (i.e., recycled) fiber pulp from sources such as, for example, newsprint, reclaimed paperboard, and office waste.

As indicated above, the polyester is spunbonded in some embodiments. As used herein, the term “spunbonded” refers to the process by which polyester is heated and extruded through fine, usually circular, capillaries of a spinnerette to form fibers, which are subsequently fed through a fiber draw unit. From the fiber draw unit, the fibers are spread onto a surface in a uniform, random manner, where they are then formed into a web of material. The “spun” material is then passed between the rolls of a heated calender to bond the fibers together and form the spunbonded polyester fabric. Various patterns can be imparted to the fabric by the calender rolls, but the principle purpose of the bonding is to increase the integrity of the fabric. Bonding may also be accomplished by needling, hydroentanglement, or other methods known in the art.

In this embodiment, spunbonded polyester may comprise between 10% and 50% of the hydrophilic fabric. In another embodiment, spunbonded polyester may comprise between about 20% and 40% of the hydrophilic fabric. In yet another embodiment, spunbonded polyester may comprise about 35% of the hydrophilic fabric. In an embodiment, the wood pulp may comprise between about 50% and 85% of the hydrophilic fabric. In another embodiment, the wood pulp may comprise between about 60% and 75% of the hydrophilic fabric. In yet another embodiment, the wood pulp may comprise about 65% of the hydrophilic fabric. In a particular embodiment, the hydrophilic fabric comprises about 35% spunbonded polyester and about 65% wood pulp.

The method of the present invention may utilize a continuous padding process typically used to apply dyes, such as indigo, to fabrics. As noted above, the method of the invention involves providing a first solution of at least one oligodynamic metal ion, with which the hydrophilic fabric is contacted and then precipitating or reducing the metal ion onto and/or into the structure of the fabric in order to be bound thereto without the need for a binding agent.

As used herein, the term “oligodynamic” refers to any metal ion which has the ability to control or reduce bacterial growth. Thus, an oligodynamic metal ion may reduce or prevent the growth of microorganisms or may kill existing microorganisms so as to operate as an antimicrobial agent.

In an embodiment, the oligodynamic metal ion can be an ion of any element classified as a metal within the art. In a separate embodiment, the oligodynamic metal ion can be an ion of any element classified on the periodic table as a transition metal. In a particular embodiment, the oligodynamic metal ion may be an ion of silver, gold, platinum, palladium, copper, zinc, iron, tin, cobalt, nickel, manganese, vanadium, and/or an alloy of the same.

In some embodiments, the first solution contains only one type of oligodynamic metal ion. For example, the first solution may contain only silver ions or only nickel ions. In other embodiments, the first solution contains more than one type of oligodynamic metal ion. For example, the first solution may contain both silver and nickel metal ions.

According to an embodiment of the invention, the oligodynamic metal ion can be provided in the form of a metal salt dissolved in water. Any water-soluble metal salt known in the art may be acceptable in this embodiment, provided that it does not react adversely with the other components used in the method of producing a fabric with antimicrobial properties. In a particular embodiment, the metal salt may be silver nitrate, silver acetate, silver fluoride, silver lactate, silver perchlorate, nickel chloride, nickel (II) chloride hexahydrate, nickel acetate, nickel formate, nickel iodide, nickel nitrate, nickel sulfate, nickel bromide, zinc chloride, zinc bromide, zinc iodide, zinc acetate, zinc nitrate, zinc sulfate, copper acetate, copper nitrate, copper sulfate, copper sulfide, ferric chloride, ferrous bromide, ferrous iodide, ferrous acetate, ferric nitrate, ferrous sulfate, manganese chloride, manganese bromide, manganese iodide, manganese acetate, manganese nitrate, manganese sulfate, vanadium chloride, vanadium bromide, vanadium iodide, vanadium acetate, vanadium nitrate, vanadium sulfate, gold chloride, gold bromide, gold iodide, gold acetate, gold nitrate, gold sulfate, cobalt chloride, cobalt bromide, cobalt iodide, cobalt acetate, cobalt nitrate, cobalt sulfate, palladium chloride, palladium bromide, palladium iodide, palladium acetate, palladium nitrate, palladium sulfate, platinum chloride, platinum bromide, platinum iodide, platinum acetate, platinum nitrate, platinum sulfate, tin chloride, tin bromide, tin iodide, tin acetate, tin nitrate, or tin sulfate.

In an embodiment, the concentration of the water-soluble metal salt in the first solution can be between about 0.100 g/L and about 10 g/L. In another embodiment, the concentration of the water-soluble metal salt in the first solution can be between about 0.200 g/L and about 7.5 g/L. In yet another embodiment, the concentration of the water-soluble metal salt in the first solution can be between about 0.300 g/L and about 5 g/L. In some embodiments, the concentration of the water-soluble metal salt in the first solution is about 0.400 g/L. In a particular embodiment, the concentration of the water-soluble metal salt in the first solution is about 1.500 g/L. In a different embodiment, the concentration of the water-soluble metal salt in the first solution is about 4.000 g/L.

As noted above, the first solution of the invention comprises water and oligodynamic metal ions. The solution, however, is free of basic nitrogen compounds or other solubilizers which have been used in the past as a necessary part of binding antimicrobial agents to such fibers. As used herein, the term “basic nitrogen compound” includes amines and nitrogen-containing compounds such as ammonia, monoethyl amine, dimethyl amine, trimethyl amine, monobutyl amine, diisopropyl amine, monoethanol amine, diethanol amine, ethylene diamine, morpholine, pyridine, picoline, and the like.

In the method of the invention, the hydrophilic fabric is placed into contact with the first solution. This can typically be accomplished by immersing the hydrophilic fabric in the first solution. While various apparatus can be utilized, an example of an appropriate contacting mechanism and method is to provide the first solution to a dyeing padder, which is a dyeing vat with a squeezing mechanism. The hydrophilic fabric may then be guided, or simply placed, into this first solution. In this embodiment, two or more cylinders or rollers under the level of the solution may squeeze the hydrophilic fabric to improve the absorption of the solution into the fabric. The hydrophilic fabric may then be removed from the first solution and, optionally, dried using any method known in the art.

Regardless of the method for contacting the hydrophilic fabric and the first solution, the hydrophilic fabric should be placed in contact with this first solution for a period of time that is sufficient to allow at least some of the metal ions to be absorbed into the hydrophilic fabric. The period of time sufficient to allow some of the metal ions to be absorbed into the hydrophilic fabric will depend on the type of fabric utilized and the level of antimicrobial activity desired in the final product. In some embodiments, such as when using fabric made from lightweight 100% cotton fibers, this contact may last for less than 30 seconds. In other embodiments, this contact may last for between about 30 and 60 seconds. In still other embodiments, such as when using a fabric made from 100% heavy-grade wool, this contact may last for between about 2 and 5 minutes. Similarly, the selection of the oligodynamic metal may affect the time necessary to allow the metal ions to be absorbed into the fabric. The amount of time necessary to allow the metal ions to be absorbed into the fabric can be determined on a case-by-case basis by one of skill in the art considering all the various factors necessary for producing an effective antimicrobial fabric.

In the final step of the inventive method, the metal ions are precipitated onto and/or into the structure of the fabric to produce a fabric that contains a metal salt and has antimicrobial characteristics. In an embodiment of this step, the hydrophilic fabric may be contacted with a second solution. The second solution, in an embodiment, may contain an anion that reacts with the oligodynamic metal cation and produces a sparingly soluble or insoluble salt of the oligodynamic metal. This insoluble or sparingly soluble metal salt precipitates out of solution and is retained within the hydrophilic fabric to allow it to possess the desired antimicrobial activity.

Thus, in an embodiment, the second solution contains at least water and a water-soluble salt. The water-soluble salt of the second solution can be selected from a wide range of substances. It is only necessary that the salt be soluble to some degree in water and furnish an anion that will react with the oligodynamic metal cation in the fabric, forming a water-insoluble or sparingly-water soluble salt. Compounds that can be used may include those that furnish phosphate, sulfate, chloride, bromide, iodide, sulfite, or carbonate anions, or the like. In some embodiments, the water-soluble salt of the second solution may be selected from sodium phosphate, sodium chloride, sodium carbonate, sodium sulfite, sodium sulfate, sodium bromide, sodium iodide, potassium chloride, potassium carbonate, potassium sulfate, potassium bromide, potassium iodide, potassium phosphate, potassium sulfite, calcium chloride, calcium bromide, calcium iodide, barium chloride, barium bromide, barium iodide, magnesium chloride, magnesium bromide, magnesium iodide, and the like. It is to be appreciated that the water-soluble salt selected for the second solution should be chosen with reference to the metal salt utilized in the first solution.

The water-soluble salt utilized in the second solution should be, but is not necessarily required to be, present in molar excess of the metal ion so as to allow complete utilization of the metal. In some embodiments, the salt of the second solution may be present in the solution in an amount which is between about 5 and 10 times the molecular concentration of the metal salt utilized in the first solution. This concentration is designed to achieve full precipitation of the oligodynamic metal salt.

In an embodiment, the concentration of the water-soluble salt in the second solution can be between about 1.0 g/L and about 50 g/L. In another embodiment, the concentration of the water-soluble salt in the second solution can be between about 1.0 g/L and about 5.0 g/L. In yet another embodiment, the concentration of the water-soluble salt in the second solution can be between about 7.5 g/L and about 15 g/L. In still another embodiment, the concentration of the water-soluble salt in the second solution can be between about 20 g/L and about 50 g/L. In some embodiments, the concentration of the water-soluble salt in the second solution is about 3.0 g/L. In a particular embodiment, the concentration of the water-soluble salt in the second solution is about 6 g/L. Again, the exact concentration will depend on the concentration of the metal ion in the first solution and the desired level of antimicrobial activity in the final fabric desired.

In an alternative embodiment, rather than containing a water-soluble salt designed to precipitate a metal salt out of solution, the second solution may contain water and a reducing agent that will convert the oligodynamic metal ion into a free metal. The reducing agent may comprise any water-soluble reducing agent known in the art. In some embodiments, the reducing agent may be a monosaccharide, such as glucose, or it may be formaldehyde, dextrin, oxalate, glyoxal, ascorbic acid, sorbitol, hydroxylamine, hydrazine, borohydride, dimethylamine borane, or a salt thereof.

In addition to the first solution being free of basic nitrogen compounds or other solubilizers, the second solution is also free of such compounds. Thus, the second solution does not contain ammonia, monoethyl amine, dimethyl amine, trimethyl amine, monobutyl amine, diisopropyl amine, monoethanol amine, diethanol amine, ethylene diamine, morpholine, pyridine, picoline, or the like.

The final step of the inventive method can be accomplished by immersing the hydrophilic fabric in the second solution. As described above with respect to the first solution, the second solution may also be placed in a dyeing padder, or other suitable container or vat, the hydrophilic fabric guided or placed into the second solution and, optionally, squeezed under the pressure of two or more cylinders or rollers. The hydrophilic fabric may then be removed from the second solution and dried.

Regardless of the manner in which the hydrophilic fabric contacts the second solution, the hydrophilic fabric should be placed in contact with this solution for a period of time that is sufficient to allow the salt anions to react with the metal cations to form insoluble or sparingly soluble metal salts. The period of time sufficient to allow the salt anions to react with the metal cations to form metal salts may depend on the type of fabric utilized. In some embodiments, such as when using fabric made from lightweight 100% cotton fibers, this contact may last for between about 30 and 60 seconds. In other embodiments, such as when using a fabric made from 100% heavy-grade wool, this contact may last for between about 2 and 5 minutes. Similarly, the selection of the oligodynamic metal and/or the water-soluble salt may affect the time necessary to allow the metal salts to precipitate. The amount of time necessary to allow the metal salts to precipitate can be determined on a case-by-case basis as discussed above.

The material may then be washed or rinsed with water to remove excess salts, optionally dried, and processed further according to standard methods known in the art to produce the desired finished article.

In an embodiment, the concentration of oligodynamic metal present in the hydrophilic fabric after the method of the invention has been completed is between about 50 ppm and 10,000 ppm. In another embodiment, the concentration of oligodynamic metal in the hydrophilic fabric is between about 100 ppm and 3,000 ppm. In yet another embodiment, the concentration of oligodynamic metal in the hydrophilic fabric is between about 500 ppm and 2,000 ppm.

The presence of the oligodynamic metal in the hydrophilic fabric provides a fabric that has antimicrobial activity. As used herein, “antimicrobial” or “antimicrobial activity” means that a material has sufficient antimicrobial activity, as measured by American Association of Textile and Color Chemists (MTCC) Test Method 100-2004 (“AATCC 100-2004”), to reduce an initial bacterial load by at least 90% over a 24-hour exposure period at 23-24° C. or, as measured by ASTM E-2149-01 (Determination of Antimicrobial Activity of Immobilized Antimicrobial Agents under Dynamic Contact Conditions), to reduce an initial bacterial load by at least 90% over a 24-hour exposure period at 23-24° C.

In the present invention, the antimicrobial activity of the fabric may be effective against any, or against only certain, pathogenic bacteria known in the art. In a particular embodiment, the antimicrobial activity of the fabric may be effective against any or certain of the bacteria of the genera Acinetobacter, Actinomyces, Bacilus, Bordetella, Borrelia, Brucella, Clostridium, Corynebacterium, Campylobacter, Deinococcus, Escherichia, Eubacterium, Enterobacter, Francisella, Flavobacterium, Glueonobacter, Helicobacter, Intrasporangium, Janthinobacterium, Klebsiella, Kingella, Legionella, Leptospira, Mycobacterium, Moraxella, Neisseria, Oscillospira, Proteus, Psendomonas, Providencia, Rickettsia, Salmonella, Staphylococcus, Shigella, Spirillum, Streptococcus, Treponema, Ureaplasma, Vibrio, Wolinella, Wolbachia, Xanthomonas, Yersinia, or Zoogloea,

In another particular embodiment, the antimicrobial activity of the fabric may be effective against any or certain of the bacteria of the species Acinetobacter calcoaceticus, Actinomyces israelii, Bacillus anthracis, Bacillus subtilis, Bordetella pertussis, Borrelia burgdorferi, Borrelia recurrentis, Brucella melitensis, Brucella abortus, Brucella suis, Brucella canis, Clostridium tetani, Corynebacterium diphtheriae, Campylobacter jejuni, Escherichia coli, Eubacterium alactolyticum, Enterobacter cloacae, Francisella tularensis, Flavobacterium meningosepticum, Helicobacter pylori, Klebsiella pneumoniae, Legionella pneumophilia, Leptospira interrogans, Mycobacterium tuberculosis, Moraxella catarrhalis, Moraxella lacunata, Neisseria gonorrhoeae, Neisseria meningitides, Proteus mirabilis, Proteus vulgaris, Psendomonas aeruginosa, Providencia alcalifaciens, Providencia stuartii, Providencia rettgeri, Rickettsia prowazekii, Salmonella choleraesuis, Salmonella thphimurium, Staphylococcus aureus, Staphylococcus epidermidis, Shigella dyseneriae, Spirillum minus, Streptococcus pneumoniae, Treponema pallidum, Ureaplasma urealyticum, Vibrio cholerae, Xanthomonas maltophilia, or Yersinia pestis. In a further embodiment, the antimicrobial activity of the fabric may be effective against K. pneumoniae, S. aureus, methicillin-resistant S. aureus, or vancomycin-resistant E. faecalis.

The fabric of the invention may also be useful in controlling or eliminating fungi if the appropriate metal is employed to impart the desired characteristics. In some embodiments, the fabric of the invention may be useful in controlling or eliminating any or certain fungi of the genera Aspergillus, Stachybotrys, Penicillium, Cladosporium, Alternaria, Aureobasidium, Chaetomium, Paecilomyces, Acremonium, Cephalosporium, Trichoderma, Epicoccum, Fusarium, Ulocladium, or Memnoniella. In another particular embodiment, the fabric of the invention may be useful in controlling or eliminating any or certain fungi of the species Aspergillus niger, Aspergillus versicolor, Aspergillus flavus, Aspergillus fumigatus, or Stachybotrys chartarum.

The fabric of the invention may also be useful in controlling or eliminating viruses if the appropriate metal is employed to impart the desired characteristics. In some embodiments, the fabric of the invention may be useful in controlling or eliminating any or certain viruses selected from the group parvovirus, influenza, rhinovirus, coxsackievirus, echovirus, togavirus, reovirus, adenovirus, orthomyxovirus, coronavirus, morbillivirus, varicella-zoster, arenavirus, parainfluenza, filovirus, respiratory syncytial virus, poxvirus, or paramyxovirus.

As noted, the fabrics produced according to the invention are rendered substantially self-sterilizing. In a particular embodiment, the fabrics produced according to the method of the invention have an antimicrobial effectiveness that results in a reduction in microbial activity of at least 90%. In another embodiment, the fabrics produced according to the method of the invention have an antimicrobial effectiveness that results in a reduction in microbial activity of at least 95%. In yet another embodiment, the fabrics produced according to the method of the invention have an antimicrobial effectiveness that results in a reduction in microbial activity of at least 99%.

It has also been discovered that the precipitated metal salt or reduced free metal in the fabric is relatively difficult to remove during washings, providing a lasting self-sterilizing effect. For example, in an embodiment, the method of the present invention provides the hydrophilic fabric with antimicrobial properties for at least 10 washes. In some embodiments, the method of the present invention provides the hydrophilic fabric with antimicrobial properties for at least 15 washes. In a particular embodiment, the method of the present invention provides the hydrophilic fabric with antimicrobial properties for at least 30 washes.

In addition, the fabrics produced according to the method of the invention may have a hand and feel similar to that of an untreated fabric that had been washed in clear water and dried. As used herein, the term “hand” refers to the organoleptic feel of a fabric as the fingers experience it when moved parallel over the fabric surface. It is generally considered as a combination of both smoothness and softness. It is to be appreciated that the hand of the fabric will vary based on the type of fabric utilized in the method of the invention. For example, a cotton fabric will have a substantially different hand than a wool fabric. Regardless of the initial hand, in an embodiment of the present invention, the fabrics produced according to the method of the invention have a hand which is within 90% of its untreated hand. In another embodiment of the present invention, the fabrics produced according to the method of the invention have a hand which is within 95% of its untreated hand.

The methods of the present invention may be utilized to prepare any finished article from the fabric where antimicrobial activity is desired in the article. In certain embodiments, the finished article may be disposable or non-disposable articles, such as surgical gowns; medical scrubs; surgical masks; blood pressure cuffs; protective mattress and/or pillow covers; dust masks; operating room protective garments; surgical drapes; patient gowns; privacy curtains; bandages; surgical gauze; sterilization wraps; wound dressings; transcutaneous products; packaging materials, particularly in the food industry to deter spoilage; cleaning products such as wipes and sponges; disposable clean suits; diapers; filter media; orthopedic cast padding/stockinettes; respirators; industrial wipes; clothing; undergarments; bedding; stockings; towels; shoe linings; feminine products; shoe covers; and/or recreational fabrics, such as tent covers. It should be understood, however, that the above-listed goods are merely exemplary and that the fabric of the present invention can be used in various other applications. Once the fabric is created pursuant to the present inventive methods, one of ordinary skill can then use known methods for forming the fabric into various finished articles.

In some embodiments, the fabric of the invention is a disposable medical cloth. In some embodiments, the disposable medical cloth may be surgical gowns; medical scrubs; surgical masks; blood pressure cuffs; operating room protective garments; surgical drapes; patient gowns; privacy curtains; bandages; surgical gauze; sterilization wraps; wound dressings; transcutaneous products; disposable clean suits; filter media; orthopedic cast padding/stockinettes; respirators; or shoe covers.

In an embodiment, the invention is a fabric having antimicrobial characteristics comprising hydrophilic fibers consisting essentially of an insoluble metal salt absorbed onto and/or into the structure of said fibers. In this embodiment, the water-soluble salt for the second solution is selected such that a single metal salt precipitates into the fabric, but the anion of the soluble metal salt of the first solution and the cation of the soluble salt of the second solution remain soluble. For example, silver nitrate can be used in the first solution and sodium chloride can be used in the second solution. In this embodiment, silver chloride precipitates into the fabric and the sodium and nitrate ions remain in solution because sodium nitrate is a soluble salt. If desired, the sodium and nitrate ions can then be washed or rinsed from the fabric and the fabric utilized as necessary.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims that follow the examples. In the examples, all percentages are given on a weight basis unless otherwise indicated.

In Examples 1-7, unless otherwise indicated, inoculation of the specified organism comprises at least 106 colony forming units (cfu)/mL of that organism, suspended in saline. Plate counts were performed at zero time of inoculation and after 9 hours of incubation at 37° C.±2° C.

### Example 1

A sample of 100% cotton felt was immersed in a solution of distilled water containing 0.394 g/L silver nitrate. The felt was then removed from the solution and excess moisture was removed with pressure to obtain a total wet pick up of 200%. As used herein, the term “total wet pick up” refers to the weight of liquid absorbed by a given fabric, on a percentage basis of the original weight of the fabric. The felt was then immersed in a second solution of distilled water containing 3 g/L sodium chloride. After immersion, the felt was removed from the second solution and rinsed with water to remove residual water-soluble salts and dried.

A sample of the treated felt was subjected to MTCC 100-2004 using the organism K. pneumoniae. The treated felt demonstrated a 99.66% reduction in microbial activity. A control using untreated material demonstrated no measurable reduction.

A sample of the treated felt was also subjected to MTCC 100-2004 using the organism S. aureus. The treated felt demonstrated 99.99% reduction in microbial activity. A control using untreated material demonstrated no measurable reduction.

### Example 2

A sample of 100% cotton muslin was immersed in a solution of distilled water containing 0.394 g/L silver nitrate. The muslin was removed from the solution and excess moisture was removed with pressure to obtain a total wet pick up of 200%. The muslin was immersed in a second solution of distilled water containing 3 g/L sodium chloride. After immersion, the muslin was removed from the second solution and rinsed with water to remove residual water-soluble salts and dried.

A sample of the treated muslin was subjected to MTCC 100-2004 using the organism K. pneumoniae. The treated muslin demonstrated 99.98% reduction in microbial activity. A control using untreated material demonstrated no measurable reduction.

A sample of the treated muslin was also subjected to AATCC 100-2004 using the organism S. aureus. The treated muslin demonstrated 99.95% reduction in microbial activity. A control using untreated material demonstrated no measurable reduction.

### Example 3

A sample of non-woven composite fabric consisting of 35% spunbonded polyester and 65% wood pulp was immersed in a solution of distilled water containing 1.42 g/L silver nitrate. The fabric was removed from the solution and excess moisture was removed with pressure to obtain a total wet pick up of 200%. The fabric was immersed in a second solution of distilled water containing 3 g/L sodium chloride. After immersion, the fabric was removed from the second solution and rinsed with water to remove residual water-soluble salts and dried.

A sample of the treated non-woven fabric was subjected to ASTM E-2149-01 (Determination of Antimicrobial Activity of Immobilized Antimicrobial Agents under Dynamic Contact Conditions) using the organism methicillin-resistant S. aureus. The treated fabric demonstrated 99.14% reduction in microbial activity. A control using untreated material demonstrated no measurable reduction.

### Example 4

A sample of non-woven composite fabric consisting of 35% spunbonded polyester and 65% wood pulp was immersed in a solution of distilled water containing 1.58 g/L silver nitrate. The fabric was removed from the solution and excess moisture was removed with pressure to obtain a total wet pick up of 200%. The fabric was immersed in a second solution of distilled water containing 3 g/L sodium carbonate. After immersion, the fabric was removed from the second solution and rinsed with water to remove residual water-soluble salts and dried.

A sample of the treated non-woven fabric was subjected to MTCC 100-2004 using the organism S. aureus. The treated fabric demonstrated 99.73% reduction in microbial activity. A control using untreated material demonstrated no measurable reduction.

A sample of the treated non-woven fabric was subjected to AATCC 100-2004 using the organism vancomycin-resistant E. faecalis. The treated fabric demonstrated more than 99.99% reduction in microbial activity. A control using untreated material demonstrated no measurable reduction.

### Example 5

A sample of non-woven composite fabric consisting of 35% spunbonded polyester and 65% wood pulp was immersed in a solution of distilled water containing 4.05 g/L nickel (II) chloride hexahydrate. The fabric was removed from the solution and excess moisture was removed with pressure to obtain a total wet pick up of 200%. The fabric was immersed in a second solution of distilled water containing 6 g/L sodium carbonate. After immersion, the fabric was removed from the second solution and rinsed with water to remove residual water-soluble salts and dried.

A sample of the treated non-woven fabric was subjected to AATCC 100-2004 using the organism vancomycin-resistant E. faecalis. The treated fabric demonstrated 99.49% reduction in microbial activity. A control using untreated material demonstrated less than 1% reduction.

Another sample of the treated non-woven fabric was subjected to MTCC 100-2004 using the organism methicillin-resistant S. aureus. The treated fabric demonstrated greater than 99.99% reduction in microbial activity. A control using untreated material demonstrated no measurable reduction.

### Example 6

A sample of polyester/cotton blend (65% polyester/35% cotton) woven cloth was immersed in a solution of distilled water containing 1.58 g/L silver nitrate. The cloth was removed from the solution and excess moisture was removed with pressure to obtain a total wet pick up of 200%. The cloth was immersed in a second solution of distilled water containing 3 g/L sodium chloride. After immersion, the fabric was removed from the second solution and rinsed with water to remove residual water-soluble salts and dried.

A sample of the treated polyester/cotton blend cloth was subjected to 15 laundering cycles. The laundered sample was tested according to AATCC 100-2004 using the organism methicillin-resistant S. aureus. The treated cloth demonstrated greater than 96% reduction in microbial activity. A similarly treated but unlaundered sample tested by the same test demonstrated a 97.4% reduction in microbial activity. A control using untreated material demonstrated no measurable reduction.

Another sample of the treated polyester/cotton blend cloth was subjected to 15 laundering cycles. The laundered sample was tested according to MTCC 100-2004 using the organism vancomycin-resistant E. faecalis. The treated cloth demonstrated 96.9% reduction in microbial activity. A similarly treated but unlaundered sample tested by the same test demonstrated a 99.07% reduction in microbial activity. A control using untreated material demonstrated no measurable reduction.

### Example 7

A necktie was immersed in a solution of distilled water containing 1.42 g/L silver nitrate. The fabric was removed from the solution and excess moisture was removed with pressure to obtain a total wet pick up of 200%. The necktie was immersed in a second solution of distilled water containing 3 g/L sodium chloride. After immersion, the fabric was removed from the second solution and rinsed with water to remove residual water-soluble salts and dried.

The treated necktie was subjected to AATCC 100-2004 using the organism methicillin-resistant S. aureus. The treated necktie demonstrated 97.59% reduction in microbial activity. A control using an untreated necktie demonstrated no measurable reduction in antimicrobial activity.

The treated necktie was also subjected to AATCC 100-2004 using the organism vancomycin-resistant E. faecalis. The treated necktie demonstrated greater than 99.82% reduction in microbial activity. A control using an untreated necktie demonstrated only 5.88% reduction in antimicrobial activity.

### Example 8

A sample of non-woven composite fabric consisting of 35% spunbonded polyester and 65% wood pulp was immersed in a solution of distilled water containing 1.42 g/L silver nitrate. The fabric was removed from the solution and excess moisture was removed with pressure to obtain a total wet pick up of 200%. The fabric was immersed in a second solution of distilled water containing 3 g/L sodium chloride. After immersion, the fabric was removed from the second solution and rinsed with water to remove residual water-soluble salts and dried.

The treated non-woven fabric was incorporated into facemasks. The facemasks were tested to determine if silver from the facemask could leach into a subject's saliva during normal wear of the mask.

A solution of artificial saliva was prepared by mixing 4.2 g of sodium bicarbonate, 0.5 g of sodium chloride, and 0.2 g of potassium carbonate into 1000 mL of distilled water. The ingredients were thoroughly mixed and the final solution adjusted to a pH of 6.59.

An absorption pad was placed inside a facemask and saturated with artificial saliva. The pad was allowed to leach antimicrobial particles from the test sample for 8 hours at 38° C. and 85% relative humidity. After 8 hours, the absorption pad was removed and transferred to the Department of Chemistry at the University of South Carolina for inductively coupled plasma (ICP) spectrometry.

The absorption pads were received and marked for identification in the laboratory. They were heated at 650° C. to form an ash. The ash was then dissolved in nitric acid. The samples were filtered through a 0.45 micron filter and the solutions were diluted to 25 mL in deionized water. The samples were then analyzed by ICP using calibration plots made with certified reference materials.

Analysis of the simulated saliva after extraction by ICP spectrometry revealed that no detectable amount of silver had leached into the simulated saliva. The level of silver in the samples was less than the limits of detection, 0.003 μg/mL. This indicates that the amount of silver leached from a facemask over an 8-hour period was less than 0.075 μg.

### Example 9

A sample of non-woven composite fabric consisting of 35% spunbonded polyester and 65% wood pulp was immersed in a solution of distilled water containing 1.42 g/L silver nitrate. The fabric was removed from the solution and excess moisture was removed with pressure to obtain a total wet pick up of 200%. The fabric was immersed in a second solution of distilled water containing 3 g/L sodium chloride. After immersion, the fabric was removed from the second solution and rinsed with water to remove residual water-soluble salts and dried.

The treated non-woven fabric was incorporated into facemasks. Two different styles of masks were tested with three replicates of each. To simulate human inhalation, air was pulled under vacuum of 1 cubic feet per minute (CFM) through a capsule HEPA filter and a Concha Therm III® unit, which heat and humidify air. The temperature of the test air was set to 37±2° C. and >90% relative humidity. The air was pulled through the facemasks and any readily releasable particles drawn through a collection substrate. For this study, the collection substrate was a Pall® zefluor 3 μm polytetrafluoroethylene (PTFE) membrane. This membrane was verified to have a HEPA particle collection efficiency of (≧99.97%) when tested against dioctyl phthalate (DOP) aerosol at a particle size of 0.3 μm diameter, per Institute of Environmental Sciences recommended practice (IEST RP-021.1), “Testing HEPA and ULPA Media.”

After passing through the facemasks, the test air was then sent through a cooling system to condense and remove moisture from the air stream. The cooling system consisted of a closed-loop bath circulator connected to a stainless steel collection vessel. The cooling system was designed to protect test equipment form moisture accumulation. This condensate liquid was then submitted for ICP analysis in order to substantiate the efficacy of the particle collection with the PTFE membrane.

After 8 hours±30 minutes of exposure to simulated inhalation, which equates to a 13.548 m³ volume of air sampled (28.3 L/min×480 min=13584 L), the test was stopped and the facemasks were carefully removed and transferred into a new polyethylene container for shipment to the Department of Chemistry at the University of South Carolina for ICP analysis.

The facemasks were placed, using forceps, into an acid-washed 125 mL Erlenmeyer flask with a watch glass top and refluxed with 20 mL concentrated nitric acid at 75° C. for 4 hours. The flask was swirled every 15 minutes. After 4 hours, the volume of the flask was approximately 10 mL. The solution was transferred to a 25 mL volumetric flask and diluted to volume with 2% nitric acid.

The ICP calibration was performed with standards supplied by High Purity Standards (Charleston, S.C.) which are traceable to the National Institute of Standards and Technology. The standards were diluted in an acid matrix that matched that of the facemasks to minimize matrix interferences. The limit of detection was approximately 0.003 μg/mL (ppm) for the facemasks.

No silver was detected in the condensate liquid collection that succeeded the facemask in the air pathway. The air that passed through the facemasks contained less than 0.5 μg of silver. The silver concentration in the air stream was less than 0.032 μg/m³ or 3.2×10⁻⁵ mg/m³. Thus, the air stream contained less than 0.33% of the OSHA permissible exposure limits for silver compounds.

### Example 10

This example illustrates the effects of the fabric of the present invention on mold growth.

A sample of non-woven composite fabric consisting of 35% spunbonded polyester and 65% wood pulp was immersed in a solution of distilled water containing 1.42 g/L silver nitrate. The fabric was removed from the solution and excess moisture was removed with pressure to obtain a total wet pick up of 200%. The fabric was immersed in a second solution of distilled water containing 3 g/L sodium chloride. After immersion, the fabric was removed from the second solution and rinsed with water to remove residual water-soluble salts and dried.

The treated non-woven fabric was incorporated into a facemask. A sample of the facemask was subjected to MTCC 100-2004 using the organism Aspergillus niger (ATCC 6275). The sample demonstrated a 97.03% reduction in microbial activity. A control using untreated material demonstrated no measurable reduction.

### Example 11

This example illustrates the antimicrobial activity of silver flakes and powder with regard to the influenza virus.

Materials: Powdered and flaked silver; A/Puerto Rice/8/34 H1N1 influenza virus in allantoic fluid, from St. Jude's Children's Research Hospital (SJCRH) repository; phosphate buffered saline (PBS); and 10-day old embryonated chicken eggs.

Methods:

The virus stocks were diluted 1/1000 in sterile PBS. 300 μL aliquots of virus dilution in microcentrifuge tubes were treated as follows:

Tube A: 30 μg of silver powder added

Tube B: 30 μg of silver powder added

Tube C: 30 μg of silver flakes added

Tube D: 30 μg of silver flakes added

Tube E: no additives

Tube F: no additives

All tubes were incubated at room temperature (23° C.) for 10 minutes. After incubation, tubes A and B were centrifuged at 13,000 rpm for 5 minutes and tubes C, D, E, and F were centrifuged at 13,000 rpm for 1 minute. All tubes were then placed on ice until processed. The amount of infectious virus particles remaining in solution was titrated by egg infection dose 50 titrations EID₅₀. Ten-fold dilutions of solution were made in sterile PBS and 100 μL of each dilution was injected into each of 3 10-day-old embryonated chickens assayed for the presence of virus by hemmagglutination with 0.5% chicken erythrocytes. EID₅₀ values were determined by the method of Reed and Muench. Reed, L. J. & Muench, H., A Simple Method of Estimating Fifty Percent Endpoints, Am. J. Hyg. 27:493-497 (1938).

Results:

The amount of infections virus (in EID₅₀) remaining after treatment was as follows:

Tube A: 1×10^{6.25} (1778279)

Tube B: 1×10^{7.25} (11782794)

Mean: 1×10^{6.99} (9780536)

Tube C: 1×10^{6.25} (1178279)

Tube D: 1×10^{6.25} (1178279)

Mean: 1×10^{6.25} (1178279)

Tube E: 1×10^{8.5} (316227766)

Tube F: 1×10^{9.25} (1778279410)

Mean: 1×10^{9.02} (1047253588)

The average percentage reduction for the two treatments, as compared to control treatments, was 99.1% for the silver powder and 99.8% for the silver flakes.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred versions contained therein.

What is claimed is:

1. A method for imparting antimicrobial characteristics to hydrophilic fabric comprising the steps of:
a) providing a hydrophilic fabric;
b) providing a first solution comprising at least one oligodynamic metal ion, wherein the first solution is free of basic nitrogen compounds;
c) contacting the hydrophilic fabric with the first solution to allow the hydrophilic fabric to absorb metal ions from the first solution; and
d) precipitating the metal ions that have been absorbed by the fabric so that the hydrophilic fabric contains an oligodynamic metal salt and has antimicrobial characteristics.

2. The method of claim 1 wherein said first solution is a solution of only one type of oligodynamic metal ion.

3. The method of claim 1 wherein said oligodynamic metal ions comprise silver ions.

4. The method of claim 1 wherein said first solution is a solution of two oligodynamic metal ions.

5. The method of claim 1 wherein said hydrophilic fabric is selected from the group consisting of cotton, a cotton/polyester blend, and a composite of wood pulp and spunbonded polyester.

6. The method according to claim 1 wherein the hydrophilic fabric comprises about 65% wood pulp and about 35% spunbonded polyester.

7. The method of claim 1 wherein said precipitating step comprises removing the hydrophilic material from the first solution and contacting the hydrophilic material with a second solution.

8. The method of claim 7 wherein said second solution comprises water and a water-soluble salt which, upon contact with the hydrophilic fabric, will form a water-insoluble or sparingly-water soluble metal salt.

9. The method of claim 7 wherein said second solution comprises a water and a water-soluble salt chosen from the group consisting of sodium phosphate, sodium chloride, sodium carbonate, sodium sulfite, sodium sulfate, sodium bromide, sodium iodide, potassium chloride, potassium carbonate, potassium sulfate, potassium bromide, potassium iodide, potassium phosphate, potassium sulfite, calcium chloride, calcium bromide, calcium iodide, barium chloride, barium bromide, barium iodide, magnesium chloride, magnesium bromide, and magnesium iodide.

10. The method of claim 7 wherein the second solution is free of basic nitrogen compounds.

11. A fabric made according to the method of claim 1.

12. The fabric according to claim 11 wherein the fabric is disposable.

13. The fabric according to claim 12 wherein the fabric is a disposable medical cloth.

14. A method for imparting antimicrobial characteristics to hydrophilic fabric comprising the steps of:
a) providing a hydrophilic fabric;
b) providing a first solution comprising at least one oligodynamic metal ion, wherein the first solution is free of basic nitrogen compounds;
c) contacting the hydrophilic fabric with the first solution to allow the hydrophilic fabric to absorb metal ions from the first solution; and
d) reducing the metal ions that have been absorbed by the fabric so that the hydrophilic fabric contains free oligodynamic metal and has antimicrobial characteristics.

15. The method of claim 14 wherein said reducing step comprises removing the hydrophilic material from the first solution and contacting the hydrophilic material with a second solution.

16. The method of claim 15 wherein said second solution comprises water and a reducing agent.

17. The method of claim 16 wherein said reducing agent is selected from the group consisting of glucose, formaldehyde, dextrin, oxalate, glyoxal, ascorbic acid, sorbitol, hydroxylamine, hydrazine, borohydride, dimethylamine borane, and salts thereof.

18. A fabric made according to the method of claim 14.

19. The fabric according to claim 18 wherein the fabric is disposable.

20. The fabric according to claim 19 wherein the fabric is a disposable medical cloth.

21. A fabric having antimicrobial characteristics comprising hydrophilic fibers consisting essentially of an insoluble metal salt absorbed onto or into said fibers.

22. The fabric of claim 21 wherein the fabric is disposable.

23. The fabric of claim 22 wherein the fabric is a disposable medical cloth.

24. The fabric of claim 21 having an antimicrobial effectiveness that results in a reduction in microbial activity of at least 90%.

25. The fabric of claim 21 having an antimicrobial effectiveness that results in a reduction in microbial activity of at least 95%.

26. The fabric of claim 21 having an antimicrobial effectiveness that results in a reduction in microbial activity of at least 99%.

27. The fabric of claim 21 having a hand which is within 90% of its hand when the fabric is untreated.

28. The fabric of claim 21 having a concentration of oligodynamic metal present in the fabric of between about 50 ppm and 10,000 ppm.

29. The fabric of claim 21 having a concentration of oligodynamic metal present in the fabric of between about 100 ppm and 3,000 ppm.

30. The fabric of claim 21 having a concentration of oligodynamic metal present in the fabric of between about 500 ppm and 2,000 ppm.

31. The fabric of claim 21 wherein the fabric has an antimicrobial effect against an organism selected from the group consisting of K. pneumoniae, S. aureus, methicillin-resistant S. aureus, and vancomycin-resistant E. faecalis.
